(19) United States
(12) Patent Application Publication
(10) Pub. No.: US 2025/0387259 A1
(43) Pub. Date: Dec. 25, 2025

(54) OCULAR IMPLANTS FOR CONTROLLED DRUG RELEASE

(71) Applicant: The Regents of the University of California, Oakland, CA (US)

(72) Inventors: Lilian Davila, Merced, CA (US); GianCarlo Montiel, Merced, CA (US); Abhimanyu Tripathi, Merced, CA (US)

(21) Appl. No.: 19/245,792

(22) Filed: Jun. 23, 2025

Related U.S. Application Data
(60) Provisional application No. 63/663,794, filed on Jun. 25, 2024.

(51) Int. Cl. A61F 9/00; A61K 9/00

(52) U.S. Cl. CPC A61F 9/0017; A61K 9/0051

(57) ABSTRACT

An ocular implant is designed for controlled and sustained drug release to treat eye diseases such as age-related macular degeneration (AMD) and other degenerative conditions. The implant includes a body with an interior cavity, a first screen separating the cavity into inner and outer portions, and a second screen covering an opening in the body. A bioactive core containing a bioactive agent is housed in the cavity. The screens, with differing porosities, regulate the release rate of the agent. The implantation method involves making an incision near the target tissue, performing a blunt dissection, positioning the implant in the dissection tunnel with the tissue contact surface facing the target tissue, and securing it with sutures or tissue adhesive. This implant and method provide a minimally invasive, customizable solution for delivering therapeutic agents directly to the eye, improving treatment outcomes and patient compliance while reducing the need for frequent interventions.

### CROSS-REFERENCE TO RELATED APPLICATIONS

This application claims the benefit of, and priority to, U.S. Provisional Patent Application Serial No. 63/663,794 filed on June 25, 2024. The entire contents of the foregoing application are incorporated by reference herein.

### TECHNICAL FIELD

The present disclosure pertains to the field of ophthalmology, specifically to an ocular implant designed for controlled and sustained drug release to treat eye diseases. This disclosure focuses on enhancing the efficiency and effectiveness of drug delivery directly to the eye, thereby addressing critical needs in treating conditions such as age-related macular degeneration (AMD) and similar degenerative eye diseases.

### BACKGROUND

Traditional methods of treating eye conditions often involve direct injections into the eye, which can cause significant discomfort and carry risks of complications and infections. These methods require frequent administration by healthcare providers, leading to increased medical costs and patient discomfort. Some existing devices attempt to provide sustained drug release, but they often fall short in effectiveness, are not customizable, and can be prohibitively expensive. The development of ocular implants has seen some innovation, with designs that attempt to allow for drug storage and controlled release. However, these designs typically do not offer the necessary customization to account for individual patient needs and may not target the drug effectively to the disease site. As a result, there remains a substantial need for improvements in this technology to provide more effective, less invasive, and more accessible treatments for eye diseases.

### SUMMARY

The present disclosure provides a novel ocular implant that leverages bio-inspired, porous designs for controlled drug delivery. This new class of ocular implants is designed with adjustable porosity to manage the release rate and concentration of therapeutic agents directly to the affected areas of the eye. The implants are designed to be versatile, allowing for modifications in pore size and configuration to suit different therapeutic needs and drug types. These implants are intended to be implanted with minimally invasive procedures and are designed to remain in place for extended periods, e.g., 1-12 months, reducing the need for frequent surgical interventions. By providing a sustained release of medication, these implants aim to maintain therapeutic drug levels in the eye, potentially improving treatment outcomes and patient compliance while reducing overall treatment costs. According to one embodiment of the present disclosure, an ocular implant for controlled drug release to treat eye diseases is disclosed. The ocular implant includes a body defining an interior cavity and an opening providing access into the interior cavity. The implant also includes a first screen disposed inside the cavity and separating the cavity into an inner portion and an outer portion, the first screen having a first plurality of pores. The implant further includes a second screen covering the opening and having a second plurality of pores. The implant additionally includes a bioactive core disposed within the interior cavity. The bioactive core includes a bioactive agent, where the first screen and the second screen control the release rate of the bioactive agent from the bioactive core.

According to another embodiment of the present disclosure, a method of implanting an ocular implant for controlled drug release to treat eye diseases is disclosed. The method includes making an incision proximate to a target tissue in the eye. The method also includes inserting the ocular implant through the incision. The ocular implant includes a body defining an interior cavity and an opening providing access into the interior cavity. The implant also includes a first screen disposed inside the cavity and separating the cavity into an inner portion and an outer portion, the first screen having a first plurality of pores. The implant further includes a second screen covering the opening and having a second plurality of pores. The implant additionally includes a bioactive core disposed within the interior cavity. The bioactive core includes a bioactive agent, where the first screen and the second screen control the release rate of the bioactive agent from the bioactive core. The method additionally includes positioning the ocular implant such that the second screen is facing sclera of the eye and securing the ocular implant to the sclera using sutures or tissue adhesive.

Implementations of the above embodiments may include one or more of the following features. According to one aspect of the above embodiment, the body, the first screen, and the second screen may be formed from a biocompatible material. Porosity or permeability of the first screen may be different from porosity or permeability of the second screen. The body may be formed from a non-biodegradable polymeric composition selected from one or more of the following: silicone, polyvinyl alcohol, ethylene vinyl acetate, polylactic acid, nylon, polypropylene, polycarbonate, cellulose, cellulose acetate, polyglycolic acid, polylactic-glycolic acid, cellulose esters, polyethersulfone, acrylics, and combinations thereof. The bioactive core may be a biodegradable tablet that releases the bioactive agent through hydrolysis or enzymatic cleavage. The bioactive core may include a hydrogel that biodegrades at a controlled rate to release the bioactive agent. The body may be cylindrical or semi-cylindrical in shape. The bioactive agent may be selected from one or more of the following agents and any others that are known to those of ordinary skill in the art: antibiotics, antivirals, antifungals, anti-inflammatory agents, anti- glaucoma agents, angiogenesis inhibitors, antimetabolites, fibrinolytics, wound modulating agents, neuroprotective drugs, and angiostatic steroids.

### BRIEF DESCRIPTION OF DRAWINGS

Various embodiments of the present disclosure are described below with reference to the following figures:

FIG. 1 is a side view of an ocular implant according to an embodiment of the present disclosure;

FIG. 2 is a side, cross-sectional view of the ocular implant of FIG. 1;

FIG. 3 is a side, cross-sectional view of the ocular implant of FIG. 1 filled with a bioactive agent;

FIG. 4 is a side, cross-sectional view of an eye with disc-shaped ocular implants disposed in the anterior and posterior segments according to an embodiment of the present disclosure;

FIG. 5 is a side, cross-sectional view of an eye with frustoconical-shaped ocular implants disposed in the anterior and posterior segments according to an embodiment of the present disclosure;

FIG. 6 is a side, cross-sectional view of an eye with an elongated oval-shaped ocular implants disposed in the anterior and posterior segments according to an embodiment of the present disclosure; and

FIG. 7 is a side, cross-sectional view of an eye with a tapered ocular implant disposed in the posterior segment according to an embodiment of the present disclosure.

### DETAILED DESCRIPTION

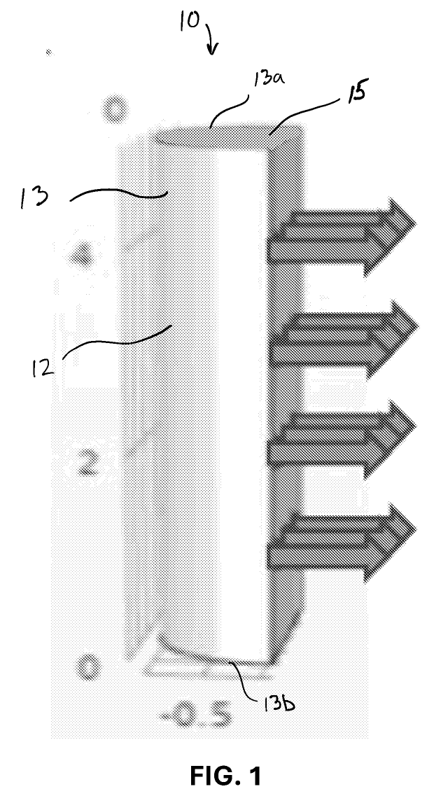

The disclosure provides several exemplary designs of bio-inspired ocular implants that utilize porous structures to control drug release efficiently. These implants are adaptable for anchoring in different areas of the eye and are designed to deliver medication over extended periods, thus reducing the frequency of medical interventions. The implants are customizable in terms of pore size and structure, allowing precise control over drug release rates and dosages. FIGS. 1-3 show an ocular implant 10 designed for implantation within the eye to provide controlled drug delivery. The implant 10 includes a body 12 formed from a plurality of walls 13 having an outer surface and an inner surface.

new paragraph...

new paragraph...

new paragraph...

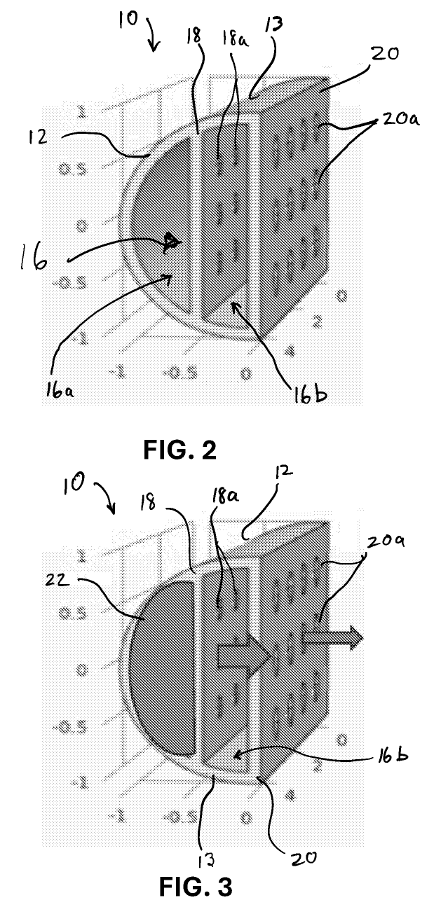

The ocular implant 10 includes an interior cavity 16 defined by the body 12, which may pass through the body from one side 13a to another side 13b, shown as top and bottom sides, respectively, in FIG. 1. The cavity 16, as illustrated in FIG. 2, may vary in size and shape but generally may occupy most of the body 12. The body 12 may have any desired, suitable shape, e.g., cylindrical or semi-cylindrical as shown in FIGS. 1-3. The body 12 may have a length of about 2 mm to about 10 mm and in another embodiment, from about 3 mm to about 5 mm. The body 12 may have a width and a height from about 0.5 mm to about 2 mm, and in another embodiment from about 1 mm to about 1.5 mm.

As illustrated in FIG. 2, the body 12 also includes a first, e.g., inner, screen 18 disposed inside the cavity 16 and a second, e.g., outer, screen 20 disposed on a plane cutting through a curved side of the body 12. The second screen 20 is disposed over an opening defined in the body 12, where the opening provides access to the cavity 16. Thus, the second screen 20 acts as an outer wall of the body 12.

The screen 18 separates the cavity 16 into two portions, a first, e.g., inner, portion 16a and a second, e.g., outer, portion 16b. The inner portion 16a is used to store a bioactive core 22 (as illustrated in FIG. 3) that releases a bioactive agent at a desired rate through the screens 18 and 20 via pores or apertures 18a and 20a respectively. The body 12 may have one or more open ends at sides 13a and/or 13b (as illustrated in FIG. 1) allowing for insertion of the core 22 into the inner portion 16a. In further embodiments, the ends may be closed with caps 15 as illustrated in FIG. 1 after insertion of the core 22.

Capping the ocular implant 10 with caps 15 has several significant effects on its functionality and efficacy. Primarily, the caps 15 provide a secure closure for the ends of the implant, ensuring that the bioactive core 22 remains securely in place within the inner portion 16a of the body 12. This containment is useful for the controlled release of bioactive agents, as it prevents any premature or uncontrolled release that could occur if the core 22 were to dislodge or shift within the cavity. Additionally, the caps 15 help maintain the structural integrity of the implant, protecting it from mechanical stresses and potential damage during handling and implantation.

Moreover, the caps 15 contribute to the overall biocompatibility of the implant 10. Being formed from biocompatible materials, they minimize the risk of adverse reactions when the implant 10 is in contact with ocular tissues. The caps 15 also enhance the ease of handling and implantation of the device. They provide a defined boundary for the core, facilitating more straightforward assembly and potentially reducing the risk of contamination during the manufacturing process. In summary, the use of caps 15 in the ocular implant not only secures and protects the bioactive core but also ensures controlled and efficient delivery of therapeutic agents, while maintaining biocompatibility and structural integrity.

The caps 15 also play a role in directing the release of the bioactive agent. By capping the ends, the implant's design ensures that the release occurs primarily through the screens 18 and 20, which have a controlled porosity. This arrangement allows for a more predictable and sustained delivery of the bioactive agents to the target tissue, enhancing the efficacy of the treatment. As illustrated in FIG. 3, since the core 22 is covered on all sides by the body 12, which is substantially impermeable, the core 22 releases the bioactive agent only though a side covered by the first screen 18. The flow of the bioactive agent is illustrated in FIGS. 1, 3, and 4 using arrows emanating from the first screen 18 toward the second screen 20 and from the second screen 20 outward, e.g., towards the tissue. Given this arrangement of the first screen 18 and the second screen 20 relative to the core 22, the direction of the flow of the bioactive agent may be controlled. The core 22 may be formed as a tablet and may further include excipients used for tableting, such as, but not limited to, fillers and lubricants. Such tablets may be produced using any suitable tableting methods known to those of ordinary skill in the art such as sublimation, roller compaction, direct compression, slugging, wet granulation, and other known methods. The bioactive agent may be distributed evenly throughout the tablet. In addition to tablets, the core 22 may be a biodegradable tablet that biodegrades at a controlled rate, releasing the bioactive agent. By way of example, such bioerosion may occur through hydrolysis or enzymatic cleavage. In other embodiments, the core 22 may be formed as a hydrogel, which may biodegrade at a controlled rate, releasing the bioactive agent. Alternatively, the hydrogel may be non-biodegradable while allowing for diffusion of the bioactive agent.

As used herein, the terms "biodegradable" and "bioabsorbable" are used with respect to a property of a material. "Biodegradable" refers to a material that is capable of being decomposed or broken down in vivo and subsequently excreted. "Bioabsorbable" is a material that is capable

of being decomposed or broken down in vivo and subsequently resorbed. Both biodegradable and bioabsorbable materials are suitable for purposes of this application and thus for simplicity, unless otherwise directed, biodegradable materials and bioabsorbable materials are collectively referred to as "biodegradable" herein. Conversely, "non-biodegradable" is a biocompatible (i.e., not harmful to living tissue) material that is not decomposed or broken down in vivo. In addition, the term "dissolution" as used in the description refers to the breakdown of both biodegradable and bioabsorbable materials.

The core 22 may include one or more bioactive agents suitable for localized delivery to a target tissue, including, without limitation, antibiotics, antivirals, and antifungals; antiallergenic agents and mast cell stabilizers; steroidal and non-steroidal anti-inflammatory agents; combinations of anti-infective and anti-inflammatory agents; decongestants; anti-glaucoma agents, including, without limitation, adrenergics, 3-adrenergic blocking agents, a-adrenergic agonists, parasympathomimetic agents, cholinesterase inhibitors, carbonic anhydrase inhibitors, and prostaglandins; combinations of anti-glaucoma agents; antioxidants; nutritional supplements; drugs for the treatment of cystoid macular edema including, without limitation, non-steroidal anti- inflammatory agents; drugs for the treatment of AMD, including, without limitation, angiogenesis inhibitors and nutritional supplements; drugs for the treatment of herpetic infections and CMV ocular infections; drugs for the treatment of proliferative vitreoretinopathy including, without limitation, antimetabolites and fibrinolytics; wound modulating agents, including, without limitation, growth factors; antimetabolites; neuroprotective drugs, including, without limitation, eliprodil; and angiostatic steroids for the treatment of diseases or conditions of the posterior segment of the eye, including, without limitation, AMD, CNV, retinopathies, retinitis, uveitis, macular edema, and glaucoma. Exemplary angiostatic steroids include 4,9(11)-Pregnadien-

17a,21-diol-3,20-dione, and 9(11)-Pregnadien-17a,21-diol-3,20-dione-21-acetate. The core 22 may also include non-active excipients to enhance the stability, solubility, penetrability, or other properties of the active agent or the drug core.

The body 12, the screens 18 and 20, and caps (if any) may be formed from a biocompatible material. The different components can be attached to the body 12 using biocompatible adhesives like silicon rubber, cyanoacrylates, thermal adhesives, epoxies, ultraviolet light cured adhesives, or any other similar materials.

In embodiments, the biocompatible material may be a non-biodegrade polymeric composition such as a homopolymer, a copolymer, straight, branched, cross-linked, or a blend of one or more polymers examples of which are provided below. Examples of polymers suitable for use in the polymeric composition include silicone, polyvinyl alcohol, ethylene vinyl acetate, polylactic acid, nylon, polypropylene, polycarbonate, cellulose, cellulose acetate, polyglycolic acid, polylactic-glycolic acid, cellulose esters, polyethersulfone, acrylics, their derivatives, and combinations thereof. The polymeric composition may also include other materials that affect its physical properties, including, but not limited to, porosity, tortuosity, permeability, rigidity, hardness, and smoothness. Exemplary materials affecting certain ones of these physical properties include plasticizers, fillers, and lubricants. The polymeric composition may include other materials that affect its chemical properties, including, but not limited to, toxicity, hydrophobicity, and interaction between the body 12 and the core 22. Body 12 may be impermeable to the bioactive agent of core 22. When body 12 is made from a generally elastic polymeric composition, the diameter of inner portion 16a may be slightly less than the diameter of core 22. This frictional fit secures core 22 within the inner portion 16a.

The screens 18 and 20 have a plurality of pores 18a and 20a, respectively. The pores 18a and 20a may have any suitable shape, e.g., circular, longitudinal, polygonal, oval, etc. The porosity and permeability of the inner screen 18 may be different from the outer screen 20. "Porosity" refers to the measure of the void spaces or pores within a material, expressed as a percentage of the total volume of the material. Porosity quantifies the extent to which a material contains pores, which can be interconnected (open porosity) or isolated (closed porosity). In the context of this disclosure, porosity is relevant to the permeability of the inner screen 18 and the outer screen 20 to the bioactive agent stored in the core 22 and affects bioactive agent release profile of the ocular implant 10 as a whole. Thus, porosity depends on a number of factors, such as the number of pores, their size, and arrangement of the pores. In embodiments, the pore size may be from about 0.25 micron to about 100 microns.

Permeability refers to the property of a material that allows fluids (liquids or gases) to pass through its porous structure. Permeability may be quantitatively expressed as the rate at which a fluid of a given viscosity flows through the material under a specified pressure gradient. Permeability is influenced by factors such as pore size, pore distribution, and the connectivity of the pore network within the material.

In one example, porosity and permeability of the inner screen 18 may be higher than that of the outer screen 20, such that the outer screen 20 acts as a limiting factor in the drug release profile of the ocular implant 10. In this embodiment, the size of pores 18a for the inner screen 18 may be from about 50 microns to about 100 microns and the size of pores 20a for the outer screen 20 may be from about 1 micron to about 40 microns. In another embodiment, porosity and permeability of the outer screen 20 may be higher than that of the inner screen 18 thereby having the inner screen 20 act as a limiting factor. In this embodiment, the pore size for the inner screen 18 may be from about 1 micron to about 40 microns and the pore size for the outer screen 20 may be from about 50 micron to about 100 microns. In further embodiments, the porosity of the inner screen 18 and the outer screen 20 may be the same. In this embodiment, the pore size for the inner screen 18 and the outer screen 20 may be from about 0.25 micron to about 100 microns or 1 micron to about 100 microns.

The structure of the ocular implant 10, in particular, the body 12 exposes the core 22 to the tissue only through the screens 18 and 20, assuming the sides are capped. Thus, the release profile of the bioactive agent may be adjusted by controlling the porosity and/or degradation of the screens 18 and 20. The porosity of the inner screen 18 may be larger than the porosity of the outer screen 20, allowing for additional modulation of the release profile. Thus, the core 22 itself has a specific release profile, which is then further modulated by a porosity or permeability of the inner screen 18, and then additionally modulated by a porosity or permeability of the outer screen 20. In embodiments, a second core (not shown) may be inserted into the outer portion 16b of the cavity 16. The second core may have one or more bioactive agents or may simply act as a buffer to further control the release rate of the bioactive agent. Second bioactive agent may be any of the exemplary bioactive agents provided above or any other desired, suitable bioactive agent. In addition, the ocular implant 10 may have multiple inner screens, each providing additional modulation of the release rate as well as further partitioning the cavity 16 into multiple portions each capable of supporting additional cores, which themselves may have one or more bioactive agents or act as release rate buffers.

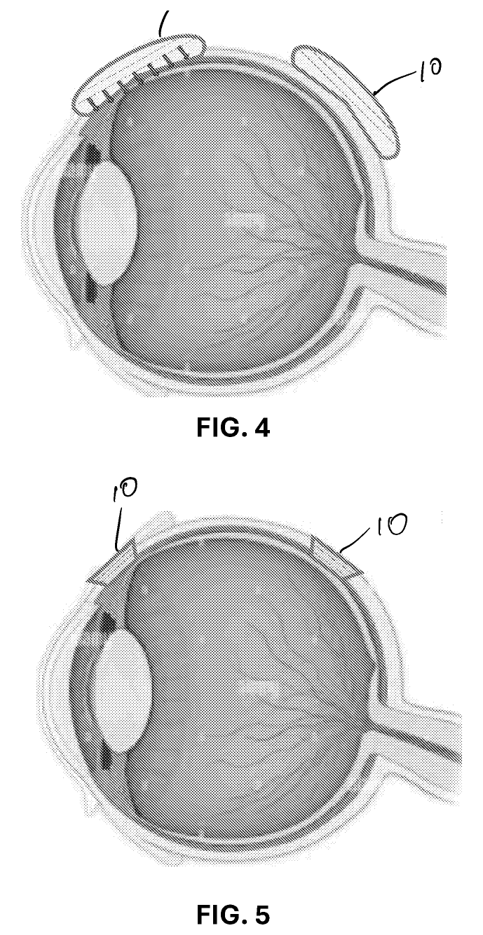

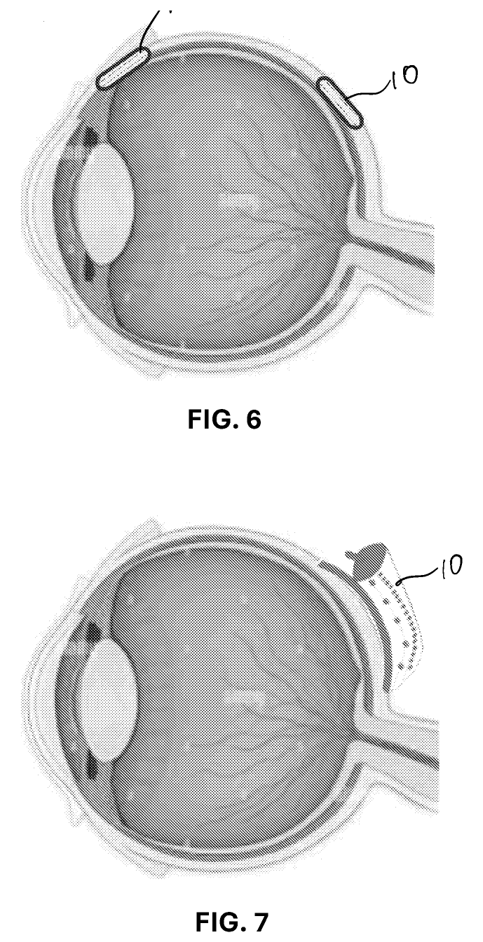

The implant 10 may have any suitable shape, such as a disc-shaped implant of FIG. 4, a frustoconical-shaped implant of FIG. 5, an elongated oval-shaped implant of FIG. 6, and a tapered- shaped implant of FIG. 7.

With reference to FIGS. 4-7, the implant 10 may be surgically placed proximate a target tissue, e.g., either anterior or posterior segments of the eye. The surgeon first makes an incision proximate the target tissue. Next, the surgeon performs a blunt dissection to a level at or near the target tissue. Once the target tissue is located, the surgeon may use forceps to hold implant 10 with tissue contact surface, i.e., the outer screen 20 (illustrated in FIGS. 2 and 3), facing the target tissue. The surgeon then introduces implant 10 into the dissection tunnel, and positions implant 10 with the outer screen 20 facing the target tissue. Once in place, the surgeon may use sutures and/or tissue adhesive to fix the implant 10 to the underlying tissue, depending on the specific tissue. After placement, the surgeon sutures the opening and may place a strip of antibiotic ointment on the surgical wound. As noted above, the implant 10 may be attached to the sclera of the eye as shown in FIGS. 4-7 such that the outer screen 20 is in contact with the sclera. Since the body 12 is otherwise closed, the bioactive agent is diffused from the core 22 through the inner and outer screens 18 and 20.

Functionally, the implant allows the controlled release of medicaments through the pores or perforations 18a and 20a in the screens 18 and 20, respectively. The perforation, aperture, or pore pattern and density are designed to regulate the flow rate and ensure sustained delivery. The medicament, which can be in the form of a tablet, powder, or slurry, is released when bodily fluids dissolve it and allow it to diffuse through the pores or perforations. The design of the implant permits variations in the shape, size, and pore or perforation pattern of the screens to accommodate different types of medicaments and treatment durations. Overall, the disclosed ocular implants provide a versatile and customizable solution for controlled drug delivery within the eye, offering the potential for effective treatment of various ocular conditions while minimizing the need for frequent medical interventions.

Alternate embodiments may be devised without departing from the spirit or the scope of the present technology. Additionally, well-known elements of embodiments of the systems, apparatuses, and methods have not been described in detail or have been omitted so as not to obscure the relevant details of the systems, apparatuses, and methods. The terminology used herein is for the purpose of describing particular embodiments only and is not intended to be limiting. The terms "comprises," "comprising," or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by "comprises ... a" does not, without more constraints, preclude the existence of additional identical elements in the process, method, article, or apparatus that comprises the element. The terms "including" and/or "having," as used herein, are defined as comprising (i.e., open language). The terms "a" or "an", as used herein, are defined as one or more than one. The term "plurality," as used herein, is defined as two or more than two. The term "another," as used herein, is defined as at least a second or more. The description may use the terms "embodiment" or "embodiments," which may each refer to one or more of the same or different embodiments. When the terms "coupled" and "connected," along with their derivatives, are used, these terms are not intended as synonyms for each other. For example, "connected" may be used to indicate that two or more elements are in direct physical or electrical contact with each other. "Coupled" may mean that two or more elements are in direct physical or electrical contact (e.g., directly coupled) or that two or more elements are not in direct contact with each other but yet still cooperate or interact with each other (e.g., indirectly coupled).

For the purposes of the description, a phrase in the form "A/B" or in the form "A and/or B" or in the form "at least one of A and B" means (A), (B), or (A and B), where A and B are variables indicating a particular object or attribute. When used, this phrase is intended to and is hereby defined as a choice of A or B or both A and B, which is similar to the phrase "and/or". Where more than two variables are present in such a phrase, this phrase is hereby defined as including only one of the variables, any one of the variables, any combination of any of the variables, and all of the variables, for example, a phrase in the form "at least one of A, B, and C" means (A), (B), (C), (A and B), (A and C), (B and C), or (A, B and C). Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in tum, in a manner that may be helpful in understanding embodiments; however, the order of description should not be construed to imply that these operations are order dependent.

As used herein, the term "about" or "approximately" applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms "substantial" and "substantially" means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., "+/-" or greater/lesser or larger/smaller), includes a variance that one skilled in the art would know to be a reasonable tolerance for the parts mentioned.

Various embodiments of the systems, apparatuses, and methods have been described, and in many of the different embodiments many features are similar. To avoid redundancy, repetitive description of these similar features may not be made in some circumstances. It shall be understood, however, that description of a first-appearing feature applies to the later described similar feature and each respective description, therefore, is to be incorporated therein without such repetition.

From the foregoing, it will be appreciated that specific embodiments of the disclosure have been described herein for purposes of illustration, but that various modifications may be made without deviating from the scope of the disclosure. Accordingly, the disclosure is not limited except as by the appended claims.

What is claimed is:

1. An ocular implant for controlled drug release to treat eye diseases, comprising:
a body defining an interior cavity and an opening providing access into the interior cavity;
a first screen disposed inside the cavity and separating the cavity into an inner portion and an outer portion, the first screen having a first plurality of pores;
a second screen covering the opening and having a second plurality of pores; and
a bioactive core disposed within the interior cavity, the bioactive core including a bioactive agent, wherein the first screen and the second screen control the release rate of the bioactive agent from the bioactive core.

2. The ocular implant of claim 1, wherein the body, the first screen, and the second screen are formed from a biocompatible material.

3. The ocular implant of claim 1, wherein porosity or permeability of the first screen is different from porosity or permeability of the second screen.

4. The ocular implant of claim 1, wherein the body is formed from a non-biodegradable polymeric composition selected from the group consisting of silicone, polyvinyl alcohol, ethylene vinyl acetate, polylactic acid, nylon, polypropylene, polycarbonate, cellulose, cellulose acetate, polyglycolic acid, polylactic-glycolic acid, cellulose esters, polyethersulfone, acrylics, and combinations thereof.

5. The ocular implant of claim 1, wherein the bioactive core is a biodegradable tablet that releases the bioactive agent through hydrolysis or enzymatic cleavage.

6. The ocular implant of claim 1, wherein the bioactive core includes a hydrogel that biodegrades at a controlled rate to release the bioactive agent.

7. The ocular implant of claim 1, wherein the body is cylindrical or semi-cylindrical in shape.

8. The ocular implant of claim 1, wherein the bioactive agent is selected from the group consisting of antibiotics, antivirals, antifungals, anti-inflammatory agents, anti-glaucoma agents, angiogenesis inhibitors, antimetabolites, fibrinolytics, wound modulating agents, neuroprotective drugs, and angiostatic steroids.

9. A method of implanting an ocular implant for controlled drug release to treat eye diseases, comprising:
making an incision proximate to a target tissue in the eye;
inserting the ocular implant through the incision, the ocular implant including:
a body defining an interior cavity and an opening providing access into the interior cavity;
a first screen disposed inside the cavity and separating the cavity into an inner portion and an outer portion, the first screen having a first plurality of pores;
a second screen covering the opening and having a second plurality of pores; and a bioactive core disposed within the interior cavity, the bioactive core including a bioactive agent, wherein the first screen and the second screen control the release rate of the bioactive agent from the bioactive core; positioning the ocular implant such that the second screen is facing sclera of the eye; and securing the ocular implant to the sclera using sutures or tissue adhesive.

10. The method of claim 9, wherein the body, the first screen, and the second screen are formed from a biocompatible material.

11. The method of claim 9, wherein porosity or permeability of the first screen is different from porosity or permeability of the second screen.

12. The method of claim 9, wherein the body is formed from a non-biodegradable polymeric composition selected from the group consisting of silicone, polyvinyl alcohol, ethylene vinyl acetate, polylactic acid, nylon, polypropylene, polycarbonate, cellulose, cellulose acetate, polyglycolic acid, polylactic-glycolic acid, cellulose esters, polyethersulfone, acrylics, and combinations thereof.

13. The method of claim 9, wherein the bioactive core is a biodegradable tablet that releases the bioactive agent through hydrolysis or enzymatic cleavage.

14. The method of claim 9, wherein the bioactive core includes a hydrogel that biodegrades at a controlled rate to release the bioactive agent.

15. The method of claim 9, wherein the body is cylindrical or semi-cylindrical in shape.

16. The method of claim 9, wherein the bioactive agent is selected from the group consisting of antibiotics, antivirals, antifungals, anti-inflammatory agents, anti-glaucoma agents, angiogenesis inhibitors, antimetabolites, fibrinolytics, wound modulating agents, neuroprotective drugs, and angiostatic steroids.
